(19) United States
(12) Patent Application Publication
(10) Pub. No.: US 2018/0325421 A1
(43) Pub. Date: Nov. 15, 2018

(54) METHOD AND DEVICE FOR MEASUREMENT OF EXHALED RESPIRATORY GAS TEMPERATURE FROM SPECIFIC REGIONS OF THE AIRWAY

(71) Applicant: Comac Medical Limited, Sofia (GB)

(72) Inventors: Todor Popov, Sofia (BG); Dimitar Popov, Sofia (BG); Andrey Popov, Sofia (BG)

(21) Appl. No.: 15/777,424

(22) PCT Filed: Nov. 17, 2016
(86) PCT No.: PCT/EP2016/077953

§ 371 Date: May 18, 2018

(30) Foreign Application Priority Data
Nov. 20, 2015 (GB) 1520496.9

(51) Int. Cl. A61B 5/087; A61B 5/00; G01K 13/02; G01K 13/12

(52) U.S. Cl. CPC A61B 5/0878; A61B 5/743; A61B 5/7405; G01K 2013/024; G01K 13/12; A61B 2562/0276; G01K 13/02

(57) ABSTRACT

An Exhaled Breath Temperature (EBT) monitor for the measurement of portions of exhaled respiratory gas temperature during a single exhalation, the monitor comprising: An inlet channel for receiving a stream of exhaled respiratory gas, a plurality of measurement chambers, temperature sensors located within more than one measurement chamber adapted for measuring the temperature of exhaled respiratory gas, a plurality of valves intermediate the air channel and each measurement chamber, configured to selectively pass portions of the gas stream in to each chamber and a control unit, configured to operate the valves and record the measurements of the temperature sensor

### FIELD OF THE INVENTION

The present invention is concerned with a method and device for the measurement of the temperature of selected portions of exhaled breath, such as may find application in medicine.

More specifically, the present invention is concerned with an apparatus useful in the performance of different medical investigations, including diagnostics and prevention and treatment of inflammatory lung and airway illnesses, such as diseases and allergies, in which analysis of the temperature of the exhaled breath may prove useful for the purpose of diagnosis and monitoring of the effect of anti-inflammatory treatments.

### BACKGROUND OF THE INVENTION

A common non-communicable disease, asthma, is linked with allergic inflammation of the airways. Evidence to this end has been collected by means of invasive methods of investigation: bronchoscopy with broncho-alveolar lavage and biopsies. Studies have established a quantitative relationship between the degree of inflammation of the airways and asthma severity, and also between a dose of an anti-inflammatory treatment and an ensuing clinical effect.

Bronchoscopy may be an uncomfortable experience for patients and also bears some risk, both during and after the investigation. Consequently, bronchoscopy is not applied routinely for the evaluation of airway inflammatory processes so as to tailor a therapy for an individual patient.

Noninvasive methods have been introduced as an alternative, for example, using measurement of nitric oxide in exhaled air, whose levels are higher in asthmatics, is complex, expensive and only suitable for use in specialized clinics.

Inflammation is a universal pathophysiological process and increased temperature is one of its five prominent features. In a patient with an inflamed airway, the inflamed airway mucosa acts to warm adjacent air to a higher level compared with the air adjacent to a comparative uninflamed mucosa. The extent of this warming of adjacent air depends upon the spread of an inflammatory region and on the level of inflammation.

The deep structures of the lung typically have temperatures representative of the body core. It is determined by the blood flowing along the rich vascular network of the alveoli, imparting its thermal energy to the alveolar gas content. The temperature of the inhaled air is tempered during its flow in and out of the branching airways, which have a separate system of blood supply deriving from the left ventricle of the heart through the bronchial arteries. As blood is the main carrier of thermal energy, processes that would modify its flow within the airway walls might be reflected in the temperature of the outgoing air, i.e. Exhaled Breath Temperature (EBT). High-precision gauging devices may pick up this signal and provide a basis for clinical inferences. As a breath is exhaled, the first part of the exhaled gas comes from the dead space of the throat and the airways, and later parts of the gas come from the alveoli themselves. Therefore accurate measurement of the temperature of different portions of an exhaled breath can give some indication of inflammation of different parts of the bronchial tree, starting from one central airway (trachea), two main bronchi, and along 17 to 23 generations of branched airways and related lung structures.

Because the thermal capacity of exhaled gas is relatively low, many devices for measurement of temperature will not reach thermal equilibrium with the gas temperature within the length of a single exhaled breath. Prior art devices have relied on collecting multiple breaths until temperature readings are stabilized, which can be referred to as “integral” EBT measurement.

Devices for the measurement of the exhaled breath temperature are described in (1) Piacentini G L, Bodini A, Zerman L, et al. ‘Relationship between exhaled air temperature and exhaled nitric oxide in childhood asthma’, Eur Respir J 2002; 20: 108-111; and (2) Paredi P, Kharitonov S A, Barnes P J. ‘Faster rise of exhaled breath temperature in asthma: a novel marker of airway inflammation?’, Am J Respir Crit Care Med 2001; 165: 181-184.

International Patent publication No. 2007/012930 discloses an EBT monitor which provides measurement of the temperature of exhaled breath as a surrogate marker of the inflammation in the intrathoracic airways, aggregated over a number of separate breaths.

U.S. Pat. No. 3,613,665 describes an air monitor with a valve chamber and temperature sensor for single-breath sampling.

European Patent EP2506757 discloses an exhaled respiratory gas temperature measurement device requiring multiple breaths, with a synchronous two-door shutter, whereby the shutter passes a portion of exhaled gas direct to atmosphere with no measurement of temperature, and a second portion of exhaled gas to a chamber for measurement of temperature, where each subsequent exhalation increase the temperature until an equilibrium is reached.

### SUMMARY OF THE INVENTION

The present invention provides a system for measuring exhaled respiratory gas temperature during a single exhalation, the system comprising:
an inlet channel for receiving a stream of exhaled respiratory gas;
a plurality of measurement chambers, each of a predetermined set of thermal characteristics;
a temperature sensor located within more than one measurement chamber adapted for measuring the temperature of exhaled respiratory gas in that measurement chamber;
a valve intermediate the inlet channel and each said measurement chamber; and
a control unit configured to operate the valves to pass predetermined portion(s) of the exhaled respiratory gas during a single exhalation of breath to respective measurement chamber(s).

The present invention also provides a system for measuring exhaled respiratory gas temperature during a single exhalation, the system comprising:
an inlet channel for receiving a stream of exhaled respiratory gas;
a plurality of measurement chambers, each of the same predetermined set of thermal characteristics;
a temperature sensor located within more than one measurement chamber adapted for measuring the temperature of exhaled respiratory gas in that measurement chamber;
a valve intermediate the inlet channel and each said measurement chamber; and
a control unit configured to operate the valves to pass two or more predetermined portion(s) of the exhaled respiratory gas corresponding to separate airway sections during a single exhalation of breath to respective measurement chamber(s).

The system may include any one or more of the following features:
a flow measurement device;
the control unit is configured to monitor the flow measurement device and calculate the volume of gas inhaled and, during exhalation, to operate the valves in order to initiate passage of predetermined portion(s) of the volume of the exhaled gas to respective measurement chamber(s);
the flow measurement device is a pressure sensor;
the temperature sensors are thermistors;
the temperature sensors are thermocouples;
the measurement chambers are constructed of a low thermal mass material;
the valves are pneumatically operated;
valves comprise an inflatable membrane within the inlet of each measurement chamber;
the control unit and valves are arranged so that one or more portions of the exhaled gas are discharged without measurement;
the system further comprises an electronic processor for processing electronic signals from the temperature sensors and a display for displaying signals from the processor;
the system is further configured to provide visual or audible prompts to the patient to instruct them to inhale and exhale at appropriate times, and to repeat the process.

The term “thermal characteristics” includes any one or more of the following parameters being thermal mass, thermal capacity, thermal conductivity, thermal resistance.

The present invention also provides a method of measuring exhaled respiratory gas temperature during a single exhalation, the method comprising:
an inlet channel receiving a stream of exhaled respiratory gas;
operating a plurality of valves, each intermediate the inlet channel and a respective separate measurement chamber of a predetermined set of thermal characteristics and that chamber having a temperature sensor adapted for measuring the temperature of exhaled respiratory gas in that measurement chamber, to pass predetermined portion(s) of the exhaled respiratory gas during a single exhalation of breath to respective measurement chamber(s).

The present invention also provides a method of measuring exhaled respiratory gas temperature during a single exhalation, the method comprising:
an inlet channel receiving a stream of exhaled respiratory gas;
operating a plurality of valves, each intermediate the inlet channel and a respective separate measurement chamber of a predetermined set of thermal characteristics the same for all the chambers and that chamber having a temperature sensor adapted for measuring the temperature of exhaled respiratory gas in that measurement chamber, to pass two or more predetermined portion(s) of the exhaled respiratory gas corresponding to separate airway sections during a single exhalation of breath to respective measurement chamber(s).

The present invention also provides a method of operating an EBT monitor for measuring exhaled respiratory gas temperature during a single exhalation, the method comprising:
detecting the start of the exhalation operation,
receiving a stream of exhaled respiratory gas at an inlet channel; and
operating valves intermediate the inlet channel and a plurality of measurement chambers each of a predetermined set of thermal characteristics to isolate predetermined portion(s) of the exhaled gas to the respective measurement chamber,
recording the output of temperature sensors located within the measurement chambers.

The method may include the following:
measuring the total volume of inhaled gas, measuring the cumulative volume of exhaled gas and operating the valves to pass predetermined fractions of the total volume of respiratory gas in an exhalation into separate measuring chambers.

In this way, the appropriate fraction of air corresponding to a section of the airways and lungs may be selected by the operator, regardless of the lung capacity of the subject or the breathing rate.

In one embodiment, the flow measurement device is a pressure sensor, and the control unit is operable to use algorithms to estimate the volume passing through the air channel by measuring the pressure difference along one section of the air channel. The pressure sensor provides an accurate indication of flow rate to partition the exhaled breath into portions as required for this application, and is simple and easy to clean. The pressure sensor may be positioned in the air inlet channel in a position where it will sense pressure differences corresponding to the direction and volumetric flow rate of the inhaled or exhaled gas.

Alternative embodiments of the invention may use thermistor temperature sensors or thermocouples.

Preferably, the measurement chambers are identical and constructed of a low thermal capacity material, in order to minimize the heat absorbed by the measurement chamber during the temperature measurement cycle.

In an embodiment of the invention, the valves are pneumatically operated. The valves may comprise an inflatable membrane within the inlet of each measurement chamber.

In one embodiment, the control unit and valves are arranged so that one or more portions of the exhaled gas are discharged without measurement.

Advantageously, the monitor may further comprise an electronic processor for processing signals from the temperature sensors and a display for displaying signals from the processor.

In one embodiment, the monitor may also provide visual and/or audible prompts to the patient to instruct them to inhale and exhale at appropriate times, and to repeat the single-exhalation process as required for a consistent measurement.

According to another aspect of the present invention, there is provided a measurement unit for analyzing portions of a stream of gas, the unit comprising an analysis block having:
an inlet channel for receiving a stream of gas;
at least two measurement chambers, each having a valve intermediate the inlet channel and that measurement chamber;
a sensor located within each said measurement chamber adapted for measuring a parameter of the gas in that measurement chamber;
wherein each said measurement chamber is connected to the inlet channel by a conduit of equal length and diameter and material to the other said chambers.

In this way, the readings recorded at the respective chambers may be directly compared.

With particular reference to the specific application of the present invention, there is provided a measurement unit for a system for measuring exhaled respiratory gas temperature during a single exhalation, the unit comprising:
an inlet channel for receiving a stream of exhaled respiratory gas;
at least two measurement chambers, each of the same predetermined set of thermal characteristics and each having a valve intermediate the inlet channel and that measurement chamber;
a temperature sensor located within each said measurement chamber adapted for measuring a parameter of the gas in that measurement chamber;
wherein each said measurement chamber is connected to the inlet channel by a conduit of equal length and diameter and material to the other said chambers.

This aspect of the present invention may comprise three or four measurement chamber/valve sets.

This aspect of the present invention also provides a method for analyzing portions of a stream of gas, the method comprising:
an inlet channel receiving a stream of gas;
measuring a parameter of the gas in each of at least two measurement chambers, each having a valve intermediate the inlet channel and that measurement chamber and a sensor located in that chamber, wherein each said measurement chamber is connected to the inlet channel by a conduit of equal length and diameter and material to the other said chambers.

With particular reference to the specific application of the present invention, this aspect of the present invention also provides a method for measuring exhaled respiratory gas temperature during a single exhalation, the method comprising:
an inlet channel receiving a stream of gas;
measuring the temperature of the gas in each of at least two measurement chambers, each having a valve intermediate the inlet channel and that measurement chamber and a temperature sensor located in that chamber, wherein each said measurement chamber is connected to the inlet channel by a conduit of equal length and diameter and material to the other said chambers.

In this aspect of the invention, the measurement chambers may have the same thermal characteristics.

### Applications of the Present Invention

The present invention is directed to an EBT monitor which allows the rapid measurement of temperature of one or more particular localized sections of the airway. The EBT monitor of the present invention is able to selectively measure one or more sections of the total airway from the lung, for example the central region and the peripheral region.

The present invention can be incorporated into an EBT monitor which provides temperature readings for the overall lung airway system, allowing comparison of EBT values measured by standard protocols (EBTst) with the EBT measured by a fractional protocol of the present invention (EBTfr), optionally for multiple regions of the airway system.

### Advantages of the Present Invention

The present invention allows a ready, quick and easy temperature measurement of a variety of sections of the airway system, such sectional measurement and analysis not having been previously possible by conventional EBT monitors. The measurement can be carried out during a single exhaled breath, which is of great advantage to the patient, who previously may have been asked to monitor breath temperatures for an extended period of time.

By collecting multiple temperature readings in separate similar measurement chambers, each reading has the same or similar errors or bias, making a reliable comparison of readings possible.

In prior art exhaled breath temperature monitors, multiple breaths were directed over the sensor in order to overcome thermal inertia of the sensor, and the measurement regime had to be repeated with different timings in order to analyse different segments of the exhaled breath. This would mean that an analysis of the early part of exhalation compared with the later alveolar breath required two sets of recordings, introducing potential errors as the patient may have changed their breathing pattern or their metabolic rate in between measurements.

The pneumatic valves that may be used do not generate heat during operation, unlike solenoid or other electronically-operated valves which would add heat to the exhaled breath and affect the recorded temperature.

The pressure type flow sensor used in some embodiments of the present invention can determine the volume of air passing accurately enough to portion the exhaled breath according to the required measurement regime, while still permitting the device to be sterilized after use.

Furthermore, an operator of the EBT monitor, during the measurement procedure of an individual patient, can readily adjust the monitor settings to pinpoint particular regions of the airway for measurement and analysis.

The ability for a medical practitioner, skilled patient or other skilled user to determine small changes in exhaled breath temperature attributable to particular sections of the airways is seen to be potentially beneficial as a means of offering early monitoring and/or control of inflammatory respiratory illness, which illnesses may be observed first by small but significant changes in exhaled breath temperatures which occur before the patient is observed to suffer acute symptoms of the illness.

In order to accurately measure the temperature of portions of the exhaled gas, a means of measurement is provided that can sample portions of the exhaled gas without introducing errors in the temperature measurement that would confound the diagnostic value. The present invention may address this issue.

### BRIEF DESCRIPTION OF THE DRAWINGS

In order that the invention may be more readily understood, a description is now given, by way of example only, reference being made to various embodiments of the present invention, in which:

FIG. 1 is a diagram of an Exhaled Breath Temperature measurement system of the present invention.

FIG. 2 is a diagram of a measurement unit which forms part of the measurement system of the present invention.

FIG. 3 is another diagram of the Exhaled Breath Temperature measurement system of the present invention showing greater detail of the control unit.

FIGS. 4A to C show layout drawings of one embodiment of the measurement unit with top, side and end views.

FIGS. 5A to F is a set of diagrams of the measurement unit illustrating the steps during operation.

### DETAILED DESCRIPTION OF THE PRESENT INVENTION

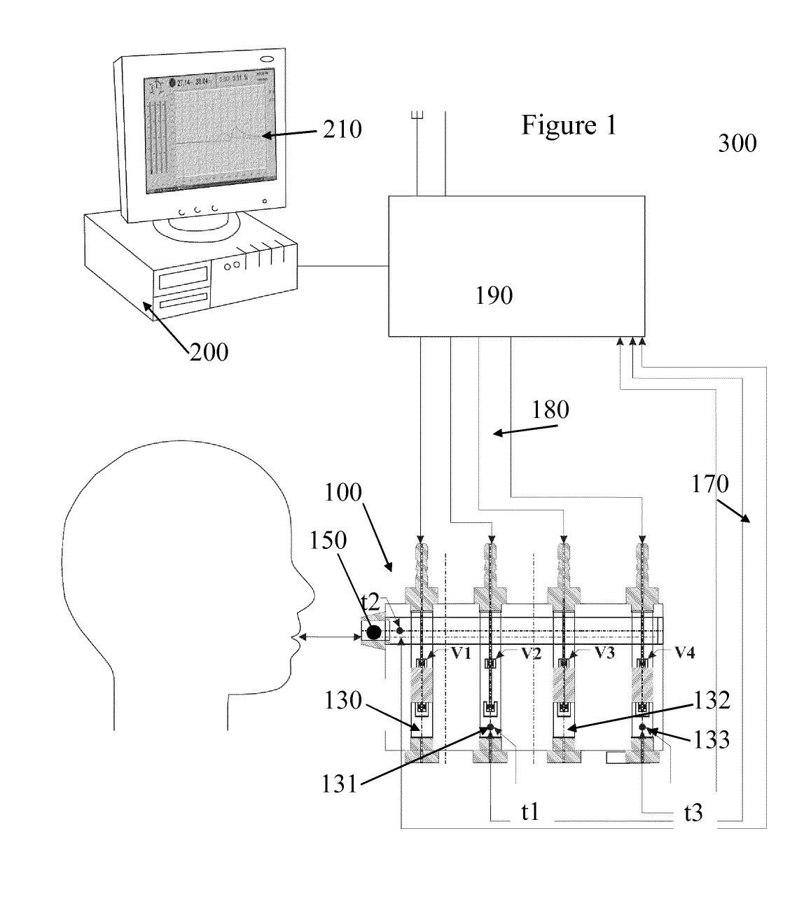

Referring now to FIG. 1, an exemplary arrangement shows a measurement unit 100, comprising measurement chambers 130-133, temperature sensors t1, t2 and t3, flow sensor 150, valves V1 to V4.

Control unit 190 comprises electronic circuitry configured to operate the valves V1 to V4 and to record the values of the temperature sensors t1 to t3 and the flow sensor 150. Control unit 190 may optionally comprise a compressed air supply 230 to operate pneumatic valves. The control unit may also include digital circuitry to convert the temperature readings into digital values and transmit them to a processor.

Control unit 190 is connected to measurement unit 100 by data cables 170, to transmit the temperature and flow sensor readings, and in this example flexible tubing 180 to operate pneumatic valves. If another type of valve is used, then appropriate connections would be required.

FIG. 1 also shows processor 200 and display 210. The processor receives digital temperature and flow readings from the control unit 190 and may collect the readings on a storage medium. Software on the processor may be configured to display temperature readings as a graph, provide medical diagnostic suggestions based on recorded temperatures and allow configuration of the control unit. The processor may be in a separate unit, for example a personal computer, or incorporated into the control unit 190.

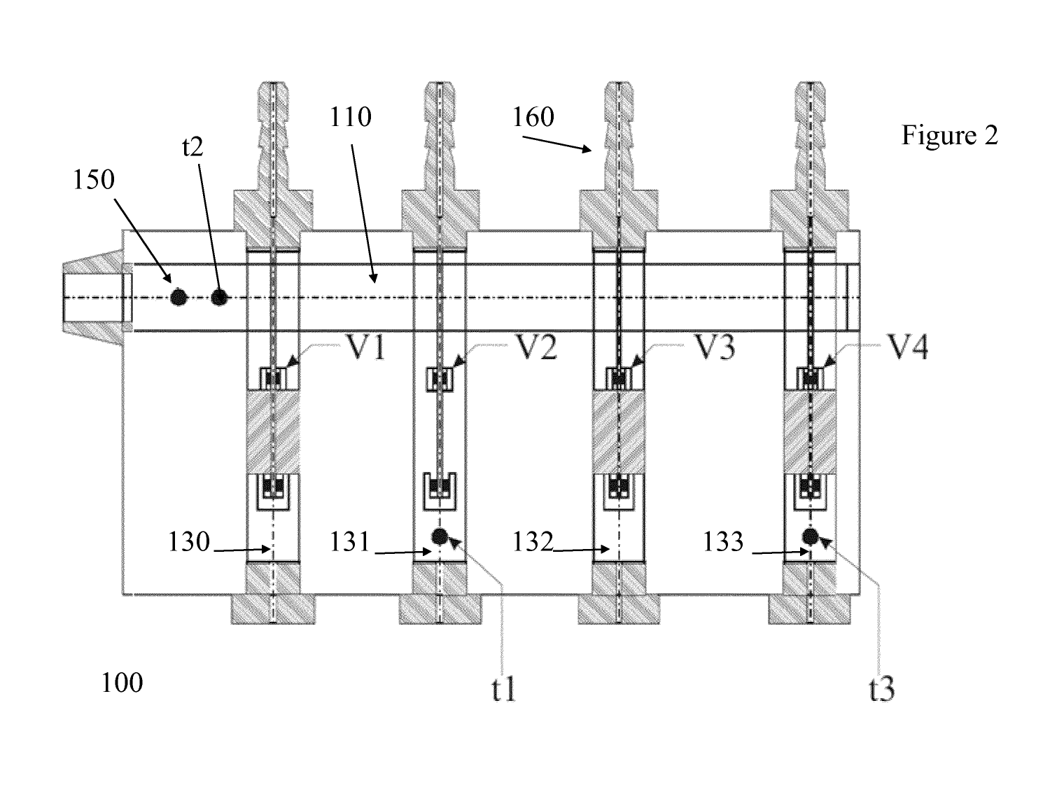

FIG. 2 shows measurement unit 100, comprising measurement chambers 130-133, temperature sensors t1, t2 and t3, flow sensor 150, valves V1 to V4. Temperature sensor t1 is positioned in measurement chamber 131, sensor t2 is located in air inlet channel 110 and sensor t3 is located in measurement chamber 133.

Temperature sensor t1 is positioned in measurement chamber 131, sensor t2 is located in air inlet channel 110 and sensor t3 is located in measurement chamber 133. Additional sensors could be installed in additional measurement chambers if required.

The valves V1 to V4 are positioned between the air inlet channel 110 and the measurement chambers 130 to 133. Valve V2 is shown open in this example while V1, V3 and V4 are shown as closed. When a valve is open, air can pass between the inlet channel and the respective measurement chamber.

In a preferred embodiment, the valves may be pneumatically operated, such as an inflatable membrane that can expand to close the top of the measurement chamber. Compressed air connectors such as 160 are shown connected to each valve. A pneumatic valve operated by compressed air at ambient temperature will cause negligible heat gain or loss in the measurement chamber and will produce no electrical interference with the temperature sensors.

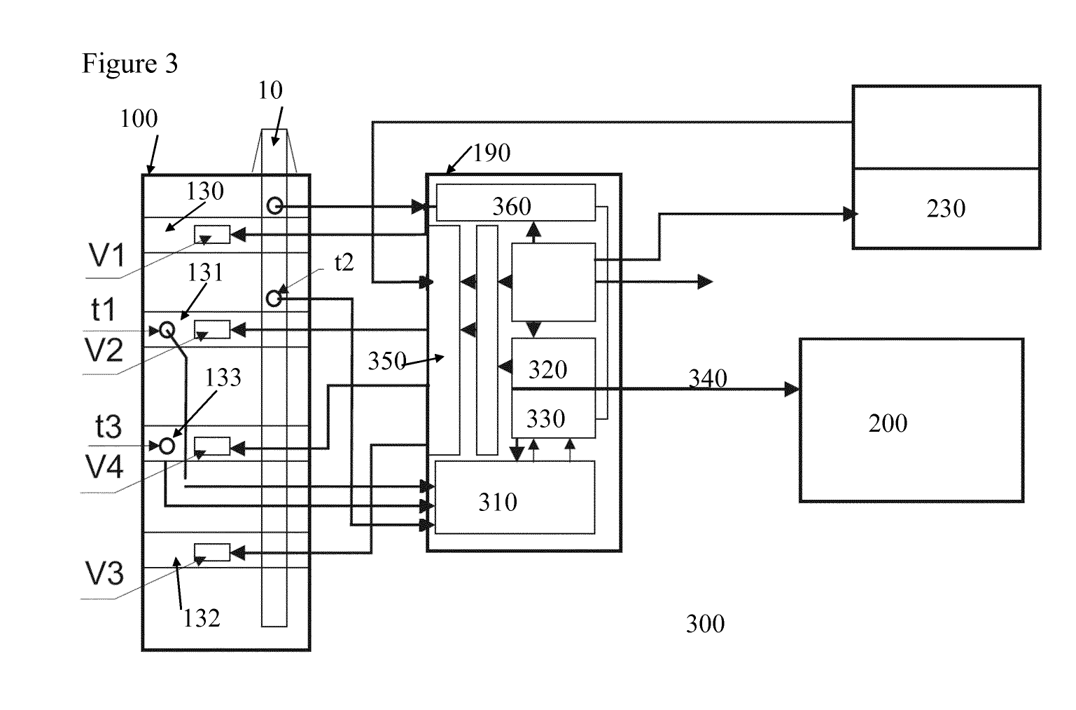

FIG. 3 shows a typical example of the exhaled breath temperature measurement system 300, showing the same referenced features as FIG. 1. In addition FIG. 3 shows a compressed air supply 230 to provide compressed air for pneumatic valves, and within control unit 190 are shown thermosensor control circuits 310, digital-to-analogue 320 and analogue-to-digital circuitry 330, a USB interface 340, valve control circuitry 350 and flow sensor control circuits 360.

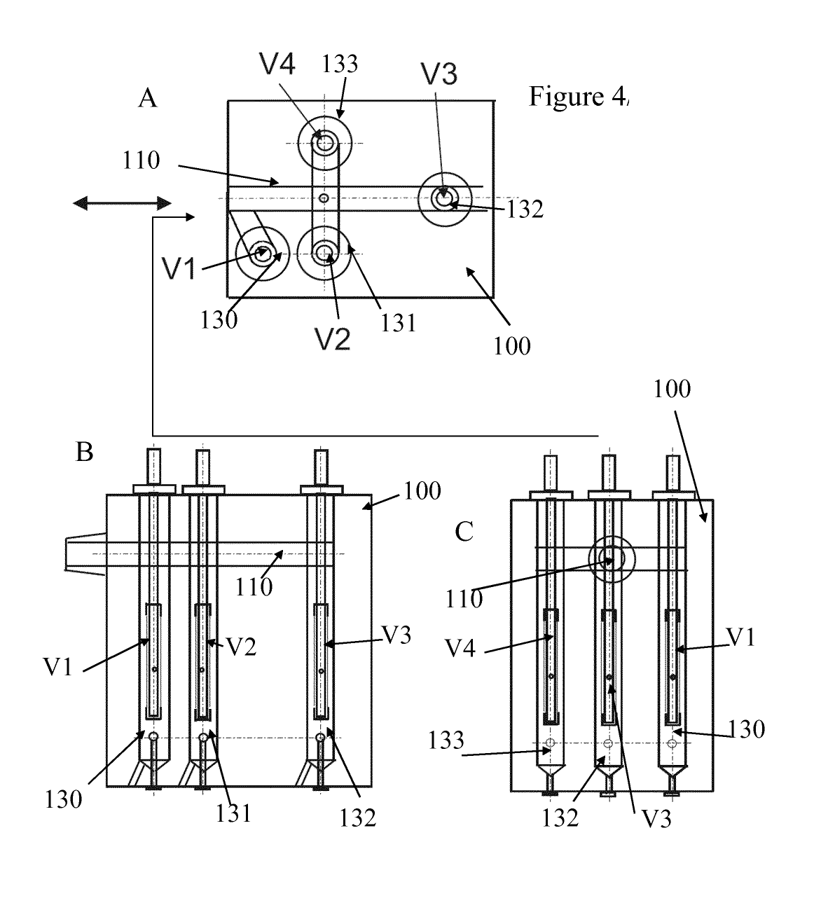

FIGS. 4A to 4C show three orthogonal projections of an exemplary embodiment of the measurement unit.

In FIG. 4A, the top view of the measurement unit 100 measurement chambers 131 and 133 are positioned opposite one another and equidistantly on either side of the air inlet channel 110. Each of measurement chambers 131 and 133 is connected to the air inlet channel by a short connection channel (not numbered) of the same diameter as the inlet channel. This arrangement ensures that exhaled gas passing to measurement chambers 131 and 133 has passed through the same length of air channel in order to minimize variations in recorded temperature due to heat absorption by the construction material of the channel.

In this exemplary embodiment, measurement chambers 130 and 132 are positioned at other locations and connected to the air inlet channel. In this embodiment measurement channels 130 and 132 do not contain temperature sensors, but are constructed with the same material and diameters as measurement channels 131 and 133 in order to ensure that the path travelled by the exhaled gas during inhalation and exhalation meets a similar flow resistance, so that determination of the volume of each portion of exhaled gas are not significantly affected by changes in pressure drop along the flow path.

Preferably, the measurement unit is constructed from a biomaterial with low thermal conductivity in order to minimize the heat transfer from the measurement chambers.

FIGS. 4B and 4C show vertical side and end views of the measurement unit, illustrating that the four measurement chambers 130 to 133 and valves V1 to V4 are arranged parallel to one another in a vertical alignment, perpendicular to the air inlet channel 110.

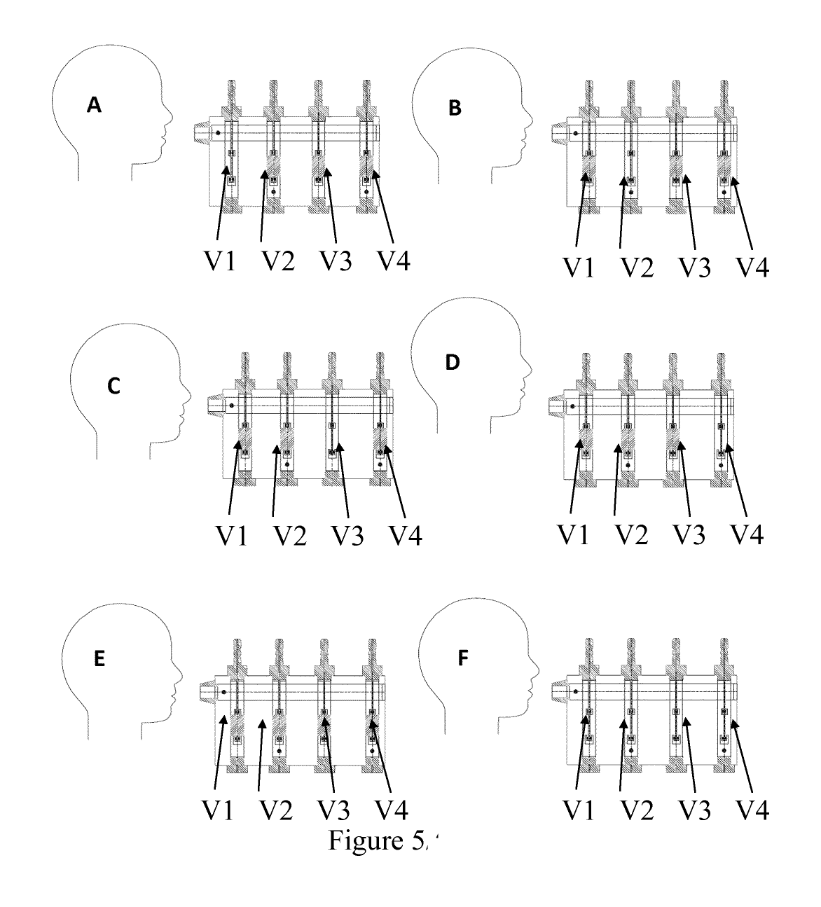

FIG. 5 shows the operation of the valves in steps A to F in an exemplary embodiment of the measurement unit 100. For clarity, only the valves V1 to V4 are referenced on the drawings, for other features refer back to FIGS. 1 to 3.

During measurement, a patient may inhale and exhale through a replaceable mouthpiece (not shown) connected to air inlet channel 110. The software in processor 200 will signal to control unit 190 when to open or close each valve, and will also record temperatures and flow from the sensors.

FIG. 5A shows that, during inhalation, valve V1 opens to allow air to pass to the patient, valves, V2, V3 and V4 are closed. Software in the processor 200 monitors the flow sensor 150 to calculate the volume of air inhaled. After completion of the inhalation, the software calculates the total volume of the inhaled air. Depending on operator settings, the software will calculate the volumes of exhaled air that are required to be passed through each measurement chamber. For example, if the operator is interested in the temperature of the first third and last third of a breath, in order to distinguish the airway temperature of the lungs from the alveolar temperature, then the software would calculate three equal volumes of one third each of the total.

To reduce errors in the temperature measurements caused by thermal inertia of the sensors themselves and the measurement chambers, preferably the volumes requiring measurement are selected to be equal.

The start of exhalation may be automatically detected by monitoring a change of direction indicated by the flow sensor, or the patient may be prompted when to exhale by visual and/or audible prompts.

FIG. 5B shows that, during exhalation, valve V2 opens up, V1, V3 and V4 are closed, while the first portion of air is exhaled; the temperature of t1 and t2 are recorded by processor 200.

FIG. 5C shows that, once the processor 200 has determined that the first portion of air has passed through the inlet channel, valve V3 is opened, V1, V2 and V4 are closed, while the second volume of air is exhaled. In this example, only the temperature t2 is recorded as the air during transition from airway to alveolar is not of interest.

FIG. 5D shows that, once the processor 200 has determined that the second portion of air has passed through the inlet channel, valve V4 opens up, V1, V2 and V3 are closed, while the third (last) portion of air is exhaled; t3 and t2 are recorded by the control unit 190.

FIG. 5E shows that, after the third volume of air has passed through the inlet channel, all valves are closed to prevent further air movement and the recorded temperatures may be displayed on the display 210 attached to the processor 200, and comparisons of interest to the operator such as the difference between t1 and t3 as well as all other derivative variables may be calculated by the processor and displayed.

FIG. 5F: After use, all valves are opened to allow the measurement chambers and the air inlet channel to reach equilibrium temperature with the atmosphere before further use.

Thus, presently the air volume is measured during a deep inspiration and the processor 200 computer drives the valve system to slice the exhaled flow into relative portions from the upper and lower airways (typically 10 to 33% of the total volume is assigned for the upper airways, and 33 to 70% of the volume for the peripheral airways).

In a variant, the air volume from the upper airways is set as an absolute value (in the range 250-350 mL), while the volume of the peripheral airways is still a proportion of the total volume to be exhaled (e.g. 70%). This may provide measurements closer to reality, to more accurately reflect the anatomic relationships in the human respiratory system: while the volume of the peripheral airways can vary widely between individuals depending on age, height, gender, respiratory morbidities (900-4000 mL), the volume of the upper airways remains relatively constant somewhere between 250 and 350 mL, the measurements closer to the anatomical peculiarities of the large and small airways. In respiratory pathology, the volume of the upper and large airways is more or less constant, while there is a lot of variability in the remainder of the bronchial tree.

The present invention in its various aspects is as set out in the appended claims.

1. A system for measuring exhaled respiratory gas temperature during a single exhalation, the system comprising:
an inlet channel for receiving a stream of exhaled respiratory gas;
a plurality of measurement chambers, each of a predetermined set of thermal characteristics;
a temperature sensor located within each measurement chamber adapted for measuring the temperature of exhaled respiratory gas in that measurement chamber;
a valve intermediate the inlet channel and each said measurement chamber; and
a control unit configured to operate the valves to pass predetermined portion(s) of the exhaled respiratory gas during a single exhalation of breath to respective measurement chamber(s).

2. A system for measuring exhaled respiratory gas temperature during a single exhalation, the system comprising:
an inlet channel for receiving a stream of exhaled respiratory gas;
a plurality of measurement chambers, each of the same predetermined set of thermal characteristics;
a temperature sensor located within more than one measurement chamber adapted for measuring the temperature of exhaled respiratory gas in that measurement chamber;
a valve intermediate the inlet channel and each said measurement chamber; and
a control unit configured to operate the valves to pass two or more predetermined portion(s) of the exhaled respiratory gas corresponding to separate airway sections during a single exhalation of breath to respective measurement chamber(s).

3. The system of claim 1 further comprising a flow measurement device.

4. The system of claim 3 wherein the control unit is configured to monitor the flow measurement device and calculate the volume of gas inhaled and, during exhalation, to operate the valves in order to initiate passage of predetermined portion(s) of the volume of the exhaled gas to respective measurement chamber(s).

5. The system of claim 3 wherein the flow measurement device is a pressure sensor.

6. The system of claim 1, wherein the temperature sensors are thermistors.

7. The system of claim 1, wherein the temperature sensors are thermocouples.

8. The system of claim 1, wherein the measurement chambers are constructed of a low thermal mass material.

9. The system of claim 1, wherein the valves are pneumatically operated.

10. The system of claim 9, wherein the valves comprise an inflatable membrane within the inlet of each measurement chamber.

11. The system of claim 1, wherein the control unit and valves are arranged so that one or more portions of the exhaled gas are discharged without measurement.

12. The system of claim 1, wherein the system further comprises an electronic processor for processing electronic signals from the temperature sensors and a display for displaying signals from the processor.

13. The system of claim 1, further configured to provide visual or audible prompts to the patient to instruct them to inhale and exhale at appropriate times, and to repeat the process.

14. A method of measuring exhaled respiratory gas temperature during a single exhalation, the method comprising:
an inlet channel receiving a stream of exhaled respiratory gas;
operating a plurality of valves, each intermediate the inlet channel and a respective separate measurement chamber of a predetermined set of thermal characteristics and that chamber having a temperature sensor adapted for measuring the temperature of exhaled respiratory gas in that measurement chamber, to pass predetermined portion(s) of the exhaled respiratory gas during a single exhalation of breath to respective measurement chamber(s).

15. The method of claim 14 wherein the predetermined set of thermal characteristics is the same for all the chambers, and wherein each of the plurality of valves pass two or more predetermined portion(s) of the exhaled respiratory gas corresponding to separate airway sections during a single exhalation of breath to respective measurement chamber(s).

16. The method of claim 14, further comprising:
detecting a start of an exhalation operation,
operating a plurality of measurement chambers each of a predetermined set of thermal characteristics to isolate predetermined portion(s) of the exhaled gas to the respective measurement chamber, and
recording the output of temperature sensors located within the measurement chambers.

17. The method of claim 14, further comprising measuring the total volume of inhaled gas, measuring the cumulative volume of exhaled gas and operating the valves to pass predetermined fractions of the total volume of respiratory gas in an exhalation into separate measuring chambers.
